(19) United States
(12) Patent Application Publication
(10) Pub. No.: US 2009/0181931 A1
(43) Pub. Date: Jul. 16, 2009

(54) ANTIVIRAL ACTIVITY OF CIDOFOVIR AGAINST ONCOLYTIC VIRUSES

(75) Inventors: Yasuo Urata, Tokyo (JP); Masaaki Ouchi, Tokyo (JP); Toshiyoshi Fujiwara, Tokyo (JP)

Correspondence Address: DARBY & DARBY P.C., P.O. BOX 770, Church Street Station, New York, NY, 10008-0770, US

(73) Assignee: ONCOLYS BIOPHARMA, INC., Tokyo (JP)

(21) Appl. No.: 12/014,947

(22) Filed: Jan. 16, 2008

(51) Int. Cl. A61K 31/675; C12N 7/06; A61P 31/12

(52) U.S. Cl. 514/86; 435/238

(57) ABSTRACT

The present invention relates to pyrimidine compounds and their use as pharmacologically active agents capable of suppressing and inhibiting viruses (e.g., oncolytic viruses). The subject compounds and compositions are particularly useful in treating and suppressing human oncolytic adenovirus infection.

### FIELD OF THE INVENTION

The present invention relates to pyrimidine compounds and their use as pharmacologically active agents capable of suppressing and inhibiting virus (e.g., oncolytic virus) activity. The compounds and compositions are particularly useful in treating and suppressing human oncolytic adenovirus infection.

### BACKGROUND OF THE INVENTION

Replication-competent oncolytic viruses are being developed for human cancer therapy. We previously developed an attenuated adenovirus Telomelysin (OBP-301), in which the human telomerase reverse transcriptase (hTERT) promoter element drives expression of E1A and E1B genes linked with an internal ribosome entry site, which replicates in and causes selective lysis of human cancer cells. A phase-I clinical trial of Telomelysin for various solid tumors is currently underway in the U.S. Although most patients are considered to have anti-adenovirus antibodies, the viremia may cause severe adverse events in patients received Telomelysin injection.

Cidofovir (CDV) is an acyclic nucleoside phosphonate having potent broad spectrum anti-DNA virus activity and has been approved for the treatment of many types of viruses including cytomegalovirus and adenovirus (U.S. Pat. No. 5,142,051).

### SUMMARY OF THE INVENTION

The purpose of the present invention is to provide a method for inhibiting a cytopathic effect and a viral replication of oncolytic viruses in an individual and a method for treating the oncolytic viral infection in an individual, comprising administering to said mammal an effective amount of cidofovir.

The present invention provides a method for inhibiting an oncolytic virus activity, comprising contacting a cidofovir with the virus.

The present invention further provides a method for treating an oncolytic viral infection in an individual, comprising administering to said individual an effective amount of cidofovir.

The present invention also provides a method for preventing or suppressing an oncolytic viral replication in an individual, comprising administering to said individual an effective amount of cidofovir.

In a preferred embodiment of the invention, the individual includes, but is not limited to, a mammal such as cancer patient. In a preferred embodiment of the invention, the oncolytic virus includes oncolytic adenovirus. The oncolytic adenovirus includes, but is not limited to, Telomelysin, Telomelysin-GFP and ONYX-015.

### BRIEF DESCRIPTION OF THE DRAWINGS

FIG. 1A shows inhibition of cytopathic effect of Telomelysin by CDV treatment.

FIG. 1B shows inhibition of viral E1A copy number of Telomelysin or wild-type Adeno virus (wt-Ad) by CDV treatment.

FIG. 2A shows inhibition of cytopathic effect of TelomeScan and ONYX-015 by CDV treatment.

FIG. 2B shows inhibition of viral E1A copy number of TelomeScan and ONYX-015 by CDV treatment.

### DETAILED DESCRIPTION OF THE INVENTION

The present invention is described in detail below.

The prior documents, publications, patents and other patent documents cited in this specification are all incorporated herein by reference.

### Definitions

The term “treat” or “treating” as used herein, refers to inhibiting or suppressing specific adverse events caused by infection of specific type of virus, and more specifically, refers to inhibiting or suppressing viral infection or a viral activity including, but not limited to, cytopathic activity, replicative activity, and infectivity of viruses. The specific type of virus includes, but is not limited to oncolytic virus, and oncolytic adenovirus, such as Telomelysin, Telomelysin-GFP, or ONYX-015.

As used herein, the phrase “contacting a cidofovir with a virus” is meant the administration or inoculation of effective amount of cidofovir to cells or tissue which exposed or suspected to be exposed with a virus in vitro or in vivo.

A dose of cidofovir for contacting to the virus may be from 2 mg/patient kg to 8 mg/patient kg, preferably from 3 mg/patient kg to 5 mg/patient kg.

As used herein, the term “individual” refers to an animal or a patient, in some embodiments a mammal, and in some embodiments a human, to whom treatment with the compounds or pharmaceutical compositions according to the present invention is provided. For treatment of those infections, conditions or disease states which are specific for a specific animal such as a human patient, the terms patient or host refer to that specific animal. The term “individual” includes biological samples taken from such animals.

The term “animal”, as used herein, includes humans, mouse, rat, rabbit, dog, cat, cow, horse, and other organisms.

As used herein, the term “therapeutically effective amount” means an amount of a compound of the present invention effective to inhibit viral replication and/or infection, or to yield the desired therapeutic response. The specific therapeutically effective amount will, obviously, vary with such factors as the particular condition being treated, the physical condition of the patient, the type of mammal or animal being treated, the duration of the treatment, the nature of concurrent therapy (if any), and the specific formulations employed and the structure of the compounds or its derivatives.

The term “effective amount” means the administration of cidofovir according to the present invention in an amount or concentration and for period of time including acute, sub-acute or chronic administration, which is effective within the context of its administration for causing an intended effect or physiological outcome in the treatment of oncolytic viral infection. Effective amounts of compounds, according to the present invention, include amounts which are therapeutically effective for delaying the onset of, inhibiting or alleviating the effects of the above disease states. Although effective amounts of compounds, according to the present invention, generally fall within the dosage range of about 3 mg/patient kg to about 5 mg/patient kg or more, amounts outside of these ranges, in certain instances, may be used, depending upon the final use of the composition.

As used herein, the term “an amount effective to inhibit viral replication and/or infection” refers to the amount of a compound of the invention (cidofovir) administered to an individual that results in a reduced level of viral replication and/or infection and thus a reduced amount of detectable virus in the individual, i.e., a reduction in viral titer or viral load. To determine an amount effective to inhibit viral replication and/or infection, the individual's viral load can be determined prior to treatment with a compound of the present invention and then subsequent to treatment. The level of viral replication can be quantified by any number of routine methodologies including, for example: quantifying the actual number of viral particles in a sample prior to and subsequent to compound administration, and quantifying the level of one or more viral antigen present in a sample prior to and subsequent to compound administration.

The term “pharmaceutically acceptable derivative” is used throughout the specification to describe any pharmaceutically acceptable salt or prodrug form (such as an ester, phosphate ester or salt of an ester or a related group) of a nucleoside compound which, upon administration to a patient, provides directly or indirectly the nucleoside compound or an active metabolite of the nucleoside compound. Pharmaceutically acceptable salts forms of the present compounds are also contemplated by the present invention. Pharmaceutically acceptable salts include those derived from pharmaceutically acceptable inorganic or organic bases and acids. Suitable salts include those derived from alkali metals such as potassium and sodium, alkaline earth metals such as calcium and magnesium, and ammonium among numerous other acids well known in the pharmaceutical art.

The term “cancer” has its understood meaning in the art, for example, an uncontrolled growth of tissue that has the potential to spread to distant sites of the body (i.e., metastasize). The term “tumor” is also understood in the art, for example, as an abnormal mass of undifferentiated cells within a multicellular organism. Tumors can be malignant or benign. Preferably, the inventive methods disclosed herein are used to prevent and treat malignant tumors. The term “cancer”, as used herein, includes but not limited to solid cancers in the head and neck, brain, kidney, stomach, large bowel, small intestine, colorectum, lung, liver, prostate, pancreas, esophagus, bladder, gallbladder/bile duct, squamous cell, breast, uterus, thyroid, ovary, bone, skin etc.; or leukemia, lymphoma, sarcoma, melanoma, carcinoma, mesenchymal tumor, neoplasm of the central nervous system (e.g., spinal axis tumors) or the like. Preferred are methods of treating and preventing tumor-forming cancers.

As can be seen from the results obtained by experiments described below, cidofovir exhibits anti-virus activity in vitro or in vivo, and can be used in the prevention and/or treatment of oncolytic viral infection.

### Cidofovir

The present invention as disclosed herein relates to a composition for and a method of treating oncolytic virus in a subject using a general formula I (Cidofovir):

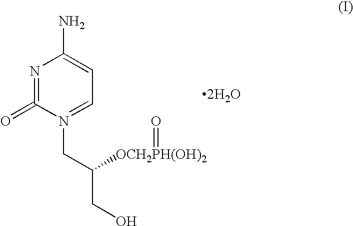

Cidofovir (CDV) is an acyclic nucleoside phosphonate having potent broad spectrum anti-DNA virus activity and has been approved for the treatment of many types of viruses including cytomegalovirus and adenovirus (U.S. Pat. No. 5,142,051).

Cidofovir is 1-[(S)-3-hydroxy-2-(phosphonomethoxy)propyl]cytosine dihydrate (HPMPC), with the molecular formula of C₈H₁₄N₃O₆P.2H₂O and a molecular weight of 315.22 (279.19 for anhydrous).

Cidofovir suppresses cytomegalovirus (CMV) replication by selective inhibition of viral DNA synthesis. Biochemical data support selective inhibition of CMV DNA polymerase by cidofovir diphosphate, the active intracellular metabolite of cidofovir. Cidofovir diphosphate inhibits herpesvirus polymerases at concentrations that are 8- to 600-fold lower than those needed to inhibit human cellular DNA polymerases alpha, beta, and gamma. Incorporation of cidofovir into the growing viral DNA chain results in reductions in the rate of viral DNA synthesis.

Cidofovir for use in the present invention is commercially available (VISTIDE ®) or can be prepared by processes known in the art, for example, described in U.S. Pat. No. 5,142,051, the disclosure of which is incorporated by reference herein their entirety.

As used herein, the term “inhibit”, “suppress” or “prevent” refers to a reduction or decrease in a quality or quantity, compared to a baseline. For example, in the context of the present invention, inhibition of viral replication refers to a decrease in viral replication as compared to baseline. In some embodiments there is a reduction of about 10%, about 20%, about 30%, about 40%, about 50%, about 60%, about 70%, about 75%, about 80%, about 85%, about 90%, about 95%, about 99%, and about 100%. Those of ordinary skill in the art can readily determine whether or not viral replication has been inhibited and to what extent. The inhibition of viral replication contributes to a reduction in the severity of the viral infection or of the symptoms of the viral infection.

As used herein, the term “about” refers to ±20%, ±15%, ±10%, or ±5% of the value.

### Pharmaceutical Composition and Method For Treating Virus Infection

In one embodiment of the invention, pharmaceutical compositions based upon the cidofovir comprise the above-described general formula I in a therapeutically effective amount for treating an oncolytic viral infection such as Telomelysin, Telomelysin-GFP or ONYX-015 infection, optionally in combination with a pharmaceutically acceptable additive, carrier or excipient. In another embodiment of the invention, pharmaceutical compositions based upon the cidofovir comprise the above-described general formula I in a therapeutically effective amount for preventing or suppressing an oncolytic viral replication such as Telomelysin, Telomelysin-GFP or ONYX-015, optionally in combination with a pharmaceutically acceptable additive, carrier or excipient. One of ordinary skill in the art will recognize that a therapeutically effective amount will vary with the infection or condition to be treated, its severity, the treatment regimen to be employed, the pharmacokinetics of the agent used, as well as the patient or host organism (animal or human) treated.

In the pharmaceutical aspect according to the present invention, cidofovir is formulated preferably in admixture with a pharmaceutically acceptable carrier. In general, it is preferable to administer the pharmaceutical composition in orally administrable form, but certain formulations may be administered via a parenteral, intravenous, intramuscular, transdermal, buccal, subcutaneous, vaginal, suppository or other route. Intravenous and intramuscular formulations are preferably administered in sterile saline. One of ordinary skill in the art may modify the formulations within the teachings of the specification to provide numerous formulations for a particular route of administration without rendering the compositions of the present invention unstable or compromising their therapeutic activity. In particular, the modification of cidofovir to render them more soluble in water or other vehicle, for example, may be easily accomplished by minor modifications (salt formulation, esterification, etc.), which are well within the ordinary skill in the art. It is also well within the ordinary artisan's skill to modify the route of administration and dosage regimen of a particular compound in order to manage the pharmacokinetics of the present compounds for maximum beneficial effect in patients.

In certain dosage forms of the pharmaceutically acceptable derivatives, the prodrug form of the compounds, especially including acylated (acetylated or other) and ether derivatives, phosphate esters and various salt forms of cidofovir, are preferred. One of ordinary skill in the art will recognize how to readily modify the present compounds to prodrug forms to facilitate delivery of active compounds to a targeted site within the host mammal or patient. The routineer also will take advantage of favorable pharmacokinetic parameters of the prodrug forms, where applicable, in delivering cidofovir to a targeted site within the host mammal or patient to maximize the intended effect of cidofovir in the treatment of oncolytic viral infections.

The amount of cidofovir included within therapeutically active formulations, according to the present invention, is an effective amount for treating the infection or condition of, in a preferred embodiment, Telomelysin, Telomelysin-GFP, or ONYX-015 infection, or preventing or suppressing the viral replication of, in a preferred embodiment, Telomelysin, Telomelysin-GFP, or ONYX-015 replication. In general, a therapeutically effective amount of cidofovir in pharmaceutical dosage form usually ranges from about 3 mg/patient kg to about 5 mg/patient kg or more, depending upon the compound used, the condition or infection treated and the route of administration.

Administration of the active compound of cidofovir may range from continuous (intravenous drip) to several oral administrations per day and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability of cidofovir from an oral route of administration. The most effective dosage form will depend upon the pharmacokinetics of cidofovir, as well as the severity of disease in the patient. Oral dosage forms are particularly preferred, because of ease of administration and prospective favorable patient compliance.

To prepare the pharmaceutical compositions according to the present invention, a therapeutically effective amount of cidofovir is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including, but not limited to, water, glycols, oils, alcohols, flavoring agents, preservatives, coloring agents and the like may be used. For solid oral preparations such as powders, tablets, capsules, and for solid preparations such as suppositories, suitable carriers and additives including, but not limited to, starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly impact the bioavailability of the compounds in the patient.

For parenteral formulations, the carrier will usually comprise sterile water or aqueous sodium chloride solution, though other ingredients including but not limited to those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed. Alternatively, cidofovir can be stored in lyophilized form to be rehydrated with an appropriate vehicle or carrier prior to use.

Liposomal suspensions (including liposomes targeted to viral antigens) may also be prepared by conventional methods to produce pharmaceutically acceptable carriers. This may be appropriate for the delivery of prodrug forms of cidofovir according to the present invention.

In particularly preferred embodiments according to the present invention, cidofovir and compositions are used to treat, prevent or delay the onset of viral infections of mammals, or prevent, delay or suppress the onset of viral replication of mammals and in particular Telomelysin, Telomelysin-GFP, or ONYX-015 infections in humans. Preferably, to treat, prevent, suppress or delay the onset of Telomelysin, Telomelysin-GFP, or ONYX-015 infections or replications, the compositions will be administered in intravenous infusion form in amounts ranging from about 3 mg/kg to 5 mg/kg or more preferably, up to one times a week. Though it will be recognized by one of ordinary skill in the art that in some instances a lower or higher dosage may be indicated.

Cidofovir may be administered alone or in combination with other antiviral agents, including other compounds of the present invention. Certain compounds according to the present invention may be effective for enhancing the biological activity of cidofovir according to the present invention by reducing the metabolism, catabolism or inactivation of other compounds and as such, are co-administered for this intended effect.

Other compounds useful in combination or alternation therapy with the compounds of the present invention include Rivavirin.

### Oncolytic Virus

The term “oncolytic virus” as used herein refers to a virus capable of exerting a cytotoxic or killing effect in vitro and in vivo to tumor cells with little or no effect on normal cells. The term “oncolytic activity” refers to cytotoxic or killing activity of a virus to tumor cells. Without wishing to be bound to any mechanism of action, the oncolytic activity is probably primarily due to cell apoptosis and to a lesser extent to plasma membrane lysis. The latter is accompanied by release of viable progeny into the cell's milieu that subsequently infect adjacent cells. The cytotoxic effects under in vitro or in vivo conditions can be detected by various means as known in the art, for example, by inhibiting cell proliferation, by detecting tumor size using gadolinium enhanced MRI scanning, by radiolabeling of a tumor, and the like.

Examples of oncolytic viruses include, but are not limited to Telomelysin, Telomelysin-GFP, ONYX-015.

### Telomelysin (OBP-301)

The recombinant virus, Telomelysin (also referred to as “OBP-301”) means a virus in which a polynucleotide comprising a promoter for human telomerase, an E1A gene, an IRES sequence and an E1B gene in this order has been integrated into its genome. The type of virus is not particularly limited, but adenoviruses are preferable from the viewpoint of safety. Among adenoviruses, type 5 adenovirus is especially preferable because of its easiness in handling, etc.

In the recombinant virus used in the present invention, the E1A gene, the IRES sequence and the E1B gene are driven by the promoter for human telomerase. Since expression of telomerase is extremely high in tumor cells compared to normal cells, the telomerase promoter is expressed in telomerase-containing tumor cells and, as a result, the recombinant virus contained in the present invention proliferates. Thus, the recombinant virus contained in the pharmaceutical composition of the present invention does not proliferate in normal cells and proliferates only in tumor cells. This means the recombinant virus is proliferated/replicated tumor cell-specifically and telomerase-specifically. As a result, viral growth causes cytotoxicity in tumor cells, by which the recombinant virus used in the present invention can kill tumor cells specifically.

The “telomerase promoter” determines the transcription initiation site for telomerase and directly regulates the frequency of transcription. Telomerase is an enzyme that maintains the length of telomeres, standing against the shortening of telomeres at the time of replication of eukaryotic chromosomes. The type of such telomerase promoter is not particularly limited, and any suitable telomerase promoter compatible with the virus to be used for the expression of a gene of interest may be used. For example, the promoter for human telomerase reverse transcriptase (hTERT) (hereinafter, referred to as “hTERT promoter”) is preferable. A great number of transcription factor-binding sequences are confirmed in a 1.4 kbp region upstream of the 5′ end of hTERT gene. This region is believed to be hTERT promoter. In particular, a 181 bp sequence located upstream of the translation initiation site is a core region important for the expression of the downstream gene. In the present invention, any sequence comprising this core region may be used. Preferably, an upstream sequence of approximately 378 bp containing the entire core region is used as hTERT promoter. It has been confirmed that this sequence of approximately 378 bp is equivalent to the 181 bp core region alone in gene expression efficiency. The nucleotide sequence of an hTERT promoter of 455 bp is shown in SEQ ID NO: 1.

The nucleotide sequence of hTERT promoter is not limited to the sequence as shown in SEQ ID NO: 1. Nucleotide sequences of nucleotides which hybridize under stringent conditions to a DNA consisting of a nucleotide sequence complementary to the DNA consisting of SEQ ID NO: 1 and have hTERT promoter activity may also be included as a sequence for hTERT promoter. Such nucleotides may be obtained from cDNA libraries or genomic libraries by known hybridization methods such as colony hybridization, plaque hybridization, Southern blotting, etc. using the polynucleotide consisting of the nucleotide sequence of SEQ ID NO: 1 or a part thereof as a probe. cDNA libraries may be prepared according to the method described in Molecular Cloning: A Laboratory Manual 2nd ed. (Cold Spring Harbor Press (1989)). Alternatively, commercial cDNA libraries or genomic libraries may be used. In the above hybridization, examples of stringent conditions include 1×SSC to 2×SSC, 0.1% to 0.5% SDS and 42° C. to 68° C. More specifically, an example may be given where prehybridization is performed at 60-68° C. for more than 30 minutes, and then washing is performed in 2×SSC, 0.1% SDS at room temperature for 5-15 minutes 4 to 6 times. For detailed procedures of hybridization, see, for example, Molecular Cloning: A Laboratory Manual 2nd ed. (Cold Spring Harbor Press (1989), in particular, Section 9.47-9.58). Further, nucleotide sequences of nucleotides which have at least 50% or more (e.g., 60% or more, 70% or more, 80% or more, 90% or more, 95% or more, 98% or more, or 99% or more) homology to the nucleotide sequence as shown in SEQ ID NO: 1 and have hTERT promoter activity may also be used as a sequence for hTERT promoter.

The reason why an E1A gene, an IRES sequence and an E1B gene are located in this order is that insertion of the IRES sequence between the E1A gene and E1B gene will results in higher replication ability of the virus when a host cell has been infected with it. E1A gene and E1B gene are genes included in E1 gene. This is one of early genes of viruses, which have early (E) genes and late (L) genes involved in their DNA replication, and encodes a protein involved in the regulation of transcription of viral genome. E1A protein encoded by E1A gene activates the transcription of a group of genes (E1B, E2, E4, etc.) necessary for the production of infectious virus. E1B protein encoded by E1B gene assists the accumulation of late gene (L gene) mRNA in the cytoplasm of the infected host cell to thereby inhibit the protein synthesis in the host cell. Thus, E1B protein promotes viral replication. The nucleotide sequences of E1A gene and E1B gene are shown in SEQ ID NO: 2 and SEQ ID NO: 3, respectively.

E1A and E1B may comprise, other than those nucleotide sequences shown in SEQ ID NO: 2 and SEQ ID NO: 3, respectively, nucleotide sequences which hybridize under stringent conditions to a DNA consisting of a nucleotide sequence complementary to the DNA consisting of SEQ ID NO: 2 or SEQ ID NO: 3 and encode a protein having E1A or E1B activity. Such nucleotide sequences may be obtained from cDNA libraries or genomic libraries by known hybridization methods such as colony hybridization, plaque hybridization, Southern blotting, etc. using the polynucleotide, or a part thereof, consisting of the nucleotide sequence of SEQ ID NO: 2 or SEQ ID NO: 3 as a probe. cDNA libraries may be prepared according to the method described in Molecular Cloning: A Laboratory Manual 2nd ed. (Cold Spring Harbor Press (1989)). Alternatively, commercial cDNA libraries or genomic libraries may be used. In the above hybridization, examples of stringent conditions include 1×SSC to 2×SSC, 0.1% to 0.5% SDS and 42° C. to 68° C. More specifically, an example may be given where prehybridization is performed at 60-68° C. for more than 30 minutes, and then washing is performed in 2×SSC, 0.1% SDS at room temperature for 5-15 minutes 4 to 6 times. For detailed procedures of hybridization, see, for example, Molecular Cloning: A Laboratory Manual 2nd ed. (Cold Spring Harbor Press (1989), in particular, Section 9.47-9.58). Further, nucleotide sequences of nucleotides which have at least 50% or more (e.g., 60% or more, 70% or more, 80% or more, 90% or more, 95% or more, 98% or more, or 99% or more) homology to the nucleotide sequence as shown in SEQ ID NO: 2 or 3 and have E1A activity (when having the above-described homology to SEQ ID NO: 2) or have E1B activity (when having the above-described homology to SEQ ID NO: 3) may also be used.

IRES (Internal Ribosome Entry Site) is a protein synthesis initiation signal specific to picornavirus. It is believed that this site serves as a ribosome-binding site because it has a complementary sequence to the 3′ terminal sequence of 18S ribosomal RNA. It is known that mRNA derived from a virus which belongs to picornaviridae is translated via this sequence. Translation efficiency from IRES sequence is high. Even from the middle of mRNA, protein synthesis is performed in a cap structure non-dependent manner. Therefore, in the virus of the present invention, both E1A gene and E1B gene (which is located downstream of the IRES sequence) are translated independently by a human telomerase promoter. By using IRES, the control of expression by a telomerase promoter is exerted on E1A gene and E1B gene independently. Therefore, compared to cases where either E1A gene or E1B gene is controlled by a telomerase promoter, viral replication can be more strictly limited to those cells having telomerase activity. An IRES sequence is shown in SEQ ID NO: 4.

IRES may comprise, other than the nucleotide sequence as shown in SEQ ID NO: 4, nucleotide sequences which hybridize under stringent conditions to a DNA consisting of a nucleotide sequence complementary to the DNA consisting of SEQ ID NO: 4 and encode a protein having IRES activity. Such nucleotide sequences may be obtained from cDNA libraries or genomic libraries by known hybridization methods such as colony hybridization, plaque hybridization, Southern blotting, etc. using the polynucleotide, or a part thereof, consisting of the nucleotide sequence of SEQ ID NO: 4 as a probe. cDNA libraries may be prepared according to the method described in Molecular Cloning: A Laboratory Manual 2nd ed. (Cold Spring Harbor Press (1989)). Alternatively, commercial cDNA libraries or genomic libraries may be used. In the above hybridization, examples of stringent conditions include 1×SSC to 2×SSC, 0.1% to 0.5% SDS and 42° C. to 68° C. More specifically, an example may be given where prehybridization is performed at 60-68° C. for more than 30 minutes, and then washing is performed in 2×SSC, 0.1% SDS at room temperature for 5-15 minutes 4 to 6 times. For detailed procedures of hybridization, see, for example, Molecular Cloning: A Laboratory Manual 2nd ed. (Cold Spring Harbor Press (1989), in particular, Section 9.47-9.58). Further, nucleotide sequences of nucleotides which have at least 50% or more (e.g., 60% or more, 70% or more, 80% or more, 90% or more, 95% or more, 98% or more, or 99% or more) homology to the nucleotide sequence as shown in SEQ ID NO: 4 and have IRES activity may also be used.

A promoter for human telomerase is located upstream of the E1 gene because such a promoter is capable of promoting viral proliferation in cells having telomerase activity.

The genes contained in the recombinant virus of the present invention may be obtained by conventional genetic engineering techniques. For example, as a genetic engineering technique, a method of nucleic acid synthesis with a commonly used DNA synthesizer may be used. Alternatively, after a genetic sequence to be used as a template is isolated or synthesized, primers specific to each gene may be designed and the genetic sequence may be amplified with a PCR apparatus (PCR method; Current Protocols in Molecular Biology, John Wiley & Sons (1987), Section 6.1-6.4); or a gene amplification method using a cloning vector may be used. One of ordinary skill in the art can readily carry out the above methods according to, for example, Molecular Cloning 2^{nd} Ed., Cold Spring Harbor Laboratory Press (1989). Purification of resultant PCR products may be performed by known methods such as a method using ethidium bromide, a method using SYBR Green I (Molecular Probes), a method with GENECLEAN (Funakoshi), QIAGEN (QIAGEN), etc. using agarose gel, a method using DEAE-cellulose filter, freeze & squeeze method or a method using a dialysis tube. When agarose gel is used, PCR products are electrophoresed on agarose gel and resultant DNA fragments are cut out from the gel and purified. If necessary, it is possible to confirm by conventional sequencing methods that an expected gene has been obtained. For example, dideoxynucleotide chain termination method (Sanger et al. (1977) Proc. Natl. Acad. Sci. USA 74: 5463) or the like may be used for this purpose. Alternatively, it is also possible to analyze the sequence with an appropriate DNA sequencer (e.g., ABI PRISM; Applied Biosystems).

Subsequently, individual genes thus obtained are ligated in a specific order. First, above genes are digested with known restriction enzymes, and the resultant DNA fragments are inserted into a known vector according to a known method for ligation. Specific examples of known vectors include pIRES vector which comprises the IRES (internal ribosome entry site in mRNA) of encephalomyocarditis virus (ECMV) and is capable of translating two open reading frames (ORFs) from one mRNA; Escherichia coli-derived plasmids (such as pCR4, pCR2, pCR2.1, pBR322, pBR325, pUC12 and pUC13); Bacillus subtilis-derived plasmids (such as pUB110, pTP5 and pC194); yeast-derived plasmids (such as pSH19 and pSH15); bacteriophages such as λ phage; animal viruses such as retrovirus, vaccinia virus and baculovirus; pA1-11, pXT1, pRc/CMV, pRc/RSV, pcDNAI/Neo and so forth. In the present invention, use of pIRES vector is preferable. With this vector, it is possible to prepare a recombinant gene comprising “a promoter for human telomerase, an E1A gene, an IRES sequence and an E1B gene” in this order by inserting necessary genes into the multi-cloning site in this order. DNA ligase may be used for ligation of DNAs. For the integration of the thus prepared recombinant gene into a virus, methods such as electroporation, the liposome method, the spheroplast method, the lithium acetate method or the like may be used.

Hereinbelow, an example where hTERT is used as human telomerase will be described specifically.

E1A gene and E1B gene may be amplified from E1 gene-expressing cells (such as 293 cells) by carrying out RT-PCR and/or DNA-PCR using primers such as E1A-S, E1A-AS, E1B-S and E1B-AS. If necessary, their sequences are confirmed using a known method such as TA cloning. DNA fragments of E1A and E1B may be cut out using known restriction enzymes.

Subsequently, a gene consisting of hTERT-E1A-IRES-E1B to be used in the present invention may be prepared by inserting individual genes into a multi-cloning site or the like of a known vector (such as pIRES vector) to give the following order: “E1A-IRES-E1B”. The sequence of hTERT promoter cut out with restriction enzymes MluI, BglIII, etc. may be inserted upstream of E1A.

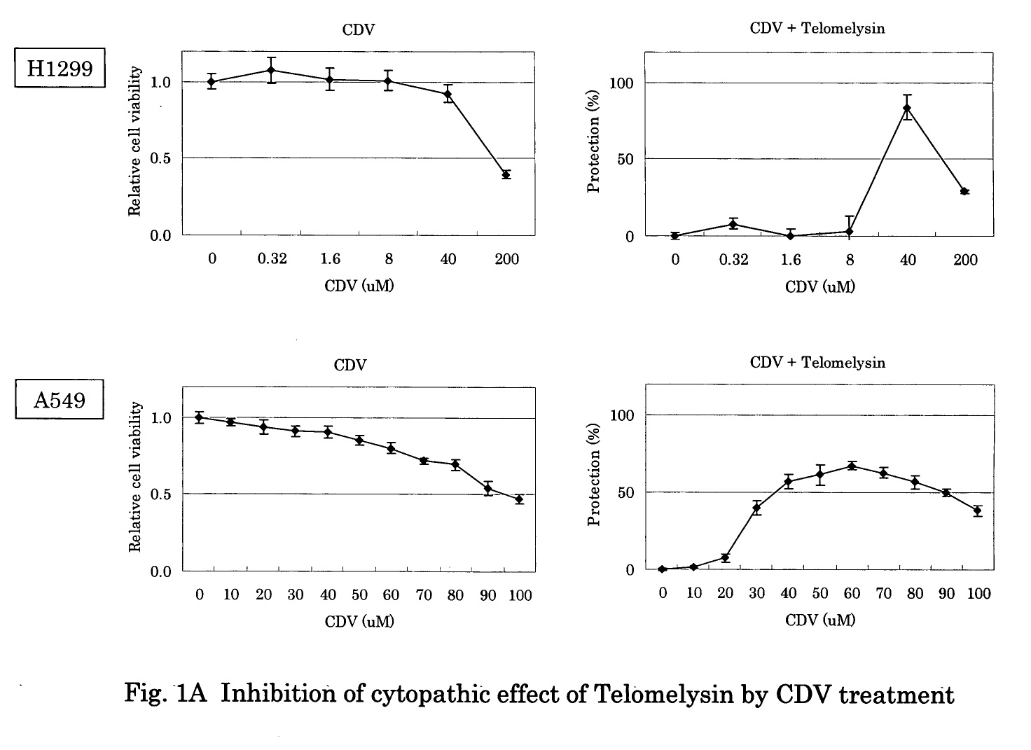

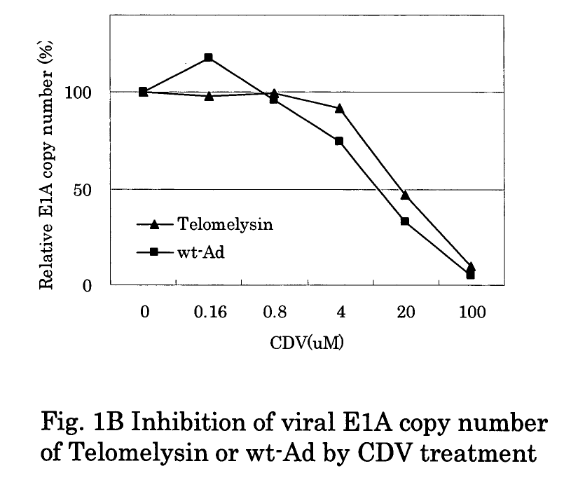

If necessary, it is also possible to remove a cytomegalovirus (CMV) promoter from a known vector such as pShuttle with appropriate restriction enzymes such as MfeI and NheI, and to insert into that site a sequence cut out from phTERT-E1A-IRES-E1B with restriction enzymes NheI and NotI. The adenovirus to be used in the present invention in which the replication cassette consisting of hTERT-E1A-IRES-E1B (FIG. 1) has been integrated is particularly designated “Telomelysin”. By expressing E1 gene necessary for proliferation of adenovirus under the control of hTERT promoter, it is possible to allow the virus to proliferate in a cancer cell-specific manner.

### Telomelysin-GFP (OBP-401)

The recombinant virus, Telomelysin-GFP (also referred to as “OBP-401”) means a recombinant virus where a replication cassette comprising a promoter from human telomerase, an E1A gene, an IRES sequence and an E1B gene in this order is integrated in E1 region of the viral genome and a labeling cassette comprising a gene encoding a labeling protein and a promoter capable of regulating the expression of the gene encoding the labeling protein is integrated in E3 region of the viral genome. As a specific example of the labeling protein, GFP may be given.

In the recombinant virus OBP-401, viral genomic regions encoding proteins with a function to promote cytotoxicity and viral dispersion (such ADP-encoding E3 region) are deleted to thereby delay the timing of cell death and facilitate identification of cancer cells by emission of GFP fluorescence or the like.

The labeling protein which constitutes the labeling cassette is a protein which emits light in those cells where the above-described virus has replicated, and is visualized. Preferably, a substance which emits fluorescence is used. Examples of such substances include, but are not limited to: green fluorescent protein (GFP) derived from luminescent jellyfish such as Aequorea victoria, enhanced-humanized GFP (EGFP) or red-shift GFP (rsGFP) which are modified variants of GFP (GFP variants). It is also possible to use yellow fluorescent protein (YFP), cyan fluorescent protein (CFP), blue fluorescent protein (BFP), or Renilla reniformis-derived GFP. A gene encoding any of these proteins may be used in the present invention.

A promoter capable of regulating the expression of the above-described gene may be any promoter as long as it is compatible with the virus to be used for the expression of the above gene of interest. Specific examples of such promoters include, but are not limited to, cytomegalovirus (CMV) promoter, hTERT promoter, SV40 late promoter, MMTV LTR promoter, RSV LTR promoter and SRα promoter. Preferably, CMV promoter or hTERT promoter may be used.

The recombinant gene contained in the labeling cassette may be obtained by conventional genetic engineering techniques. For example, as a genetic engineering technique, a method of nucleic acid synthesis with a commonly used DNA synthesizer may be used. Alternatively, after a genetic sequence to be used as a template is isolated or synthesized, primers specific to each gene may be designed and the genetic sequence may be amplified with a PCR apparatus (PCR method; Current Protocols in Molecular Biology, John Wiley & Sons (1987), Section 6.1-6.4); or a gene amplification method using a cloning vector may be used. One of ordinary skill in the art can readily carry out the above methods according to, for example, Moleculer Cloning 2^{nd} Ed., Cold Spring Harbor Laboratory Press (1989). Purification of the resultant PCR product may be performed by known methods such as a method using ethidium bromide, a method using SYBR Green I (Molecular Probes), a method with GENECLEAN (Funakoshi), QIAGEN (QIAGEN), etc. using agarose gel, a method using DEAE-cellulose filter, freeze & squeeze method or a method using a dialysis tube. When agarose gel is used, the PCT production is electrophoresed on agarose gel and resultant DNA fragments are cut out from the gel and purified. If necessary, it is possible to confirm by conventional sequencing methods that an expected gene has been obtained. For example, dideoxynucleotide chain termination method (Sanger et al. (1977) Proc. Natl. Acad. Sci. USA 74: 5463) or the like may be used for this purpose. Alternatively, it is also possible to analyze the sequence with an appropriate DNA sequencer (e.g., ABI PRISM; Applied Biosystems). Subsequently, the gene thus obtained is digested with known restriction enzymes. The recombinant gene is so designed that the resultant, digested DNA fragment (encoding the labeling protein) is located downstream of a gene fragment encoding the above-described promoter. Here, shuttle plasmid pHM11 or the like may be used as a plasmid. The two genes (for the labeling protein and the promoter) are ligated with DNA ligase and inserted into a vector to thereby prepare a recombinant gene for the labeling cassette.

As a known vector, pShuttle vector, Escherichia coli-derived plasmid (such as pCR4, pCR2, pCR2.1, pBR322, pBR325, pUC12 or pUC13); Bacillus subtilis-derived plasmid (such as pUB110, pTP5 or pC194); yeast-derived plasmid (such as pSH19 or pSH15); bacteriophage such as λ phage; animal viruse such as retrovirus, vaccinia virus or baculovirus; pA1-11, pXT1, pRc/CMV, pRc/RSV, pcDNAI/Neo or the like may be used.

Subsequently, a recombinant gene comprising the above described replication cassette and labeling cassette is cut out with appropriate restriction enzymes and inserted into an appropriate virus expression vector, to thereby prepare a recombinant virus. Examples of virus expression vectors include adenovirus, retrovirus, vaccinia virus and baculovirus. As described above, adenovirus (in particular, type 5 adenovirus) is preferably used. For integration of the cassettes into virus, methods such as electroporation, the liposome method, the spheroplast method or the lithium acetate method may be used.

In the present invention, specifically, the recombinant gene may be prepared by inserting CMV-EGFP-SV40P (A) from pEGFP-N1 (CLONTECH) into shuttle plasmid pHM11, and inserting Csp45I fragment of this plasmid into ClaI site of pShuttle vector in which phTERT-E1A-IRES-E1B has been integrated.

In the present invention, specifically, a sequence of a necessary region may be cut out with restriction enzymes from the recombinant gene as prepared above, and inserted into a viral DNA such as Adeno-X Viral DNA using a commercial kit such as Adeno-X Expression System (CLONTECH) (the resultant product is designated “AdenoX-hAIB”).

This AdenoX-hAIB is linearized with a known restriction enzyme and then transfected into cultured cells, such as 293 cells, to thereby prepare an infectious recombinant virus.

### ONYX-015

ONYX-015 is an E1B 55 kDa gene-deleted adenovirus. The virus contains a deletion between nucleotides 2496 and 3323 in the E1B region encoding the 55 kDa protein. ONYX-015 was grown in the human embryonic kidney cell line HEK293 and was purified by CsCl gradient ultracentrifugation as described previously (Bischoff J. R. et al., An adenovirus mutant that replicates selectively in p53-deficient human tumor cells. Science (Washington, D.C.), 274: 373-376, 1996).

ONYX-015 is an attenuated adenovirus that replicates in and causes selective lysis of cancer cells. The normal function of the 55 kDa protein of E1B is to bind to and inactivate p53 protein in infected cells. Because ONYX-015 lacks this protein, it is unable to replicate efficiently in cells with functional p53 because it cannot inactivate p53. However, it can replicate effectively in cells lacking functional p53, causing cytopathic effects. This approach has potential advantages over the traditional therapies of chemotherapy and radiotherapy because of the possibility of targeting tumors at a molecular level while leaving normal tissue relatively unaffected.

The following examples are set forth to further describe the invention but are in no way meant to be construed as limiting the scope thereof.

### EXAMPLES

In the present invention, we examined whether CDV can exhibit the antiviral activity against oncolytic viruses such as Telomelysin, TelomeScan (OBP-401), and ONYX-015 by XTT cell viability assay and real-time quantitative PCR assay.

### Example 1

### 1. Test Materials

Test materials used in the present invention are given below.

Test Materials

| | |
|---|---|
| Virus | |
| Telomelysin (OBP-301) | (5.0 × 10¹⁰ PFU/ml) |
| Wild-type adenovirus | (1.0 × 10¹¹ PFU/mL) |
| (OBP-202) | Commercially available from Introgen |
| Telomelysin-GFP | (2.7 × 10¹⁰ PFU/ml) |
| (OBP-401) | |
| ONYX-015 | (2.0 × 10¹¹ PFU/ml) |
| Cell lines | |
| H1299 | NSCLC, Purchased from ATCC |
| A549 | Lung cancer, Obtained from Okayama |
| | university |
| Other materials | |
| VISTIDE ™ (cidofovir | Purchased from Gilead Sciences, |
| injection) | Cat#: NDC61958-0101-1 |
| RPMI-1640 | SIGMA, Cat#: R8758, |
| D-MEM/F-12 | SIGMA, Cat#: D8062, |
| D-PBS | SIGMA, Cat#: D8537, |
| Trypsin/EDTA | SIGMA, Cat#: T4049, |
| PG/SM | SIGMA, Cat#: P4333, |
| FBS | CCT, Cat#: CC3008-502, |
| Cell Proliferation Kit II | Roche, Cat#: 11465015001, |
| QIAamp ® DNA Mini Kit | QIAGEN, Cat#: 51306, |
| RNaseA | Invitrogen, Cat#: 12091-039, |
| Ethanol | Nacalai tesque, Cat#: 14713-95, |
| LightCycler ™ Faststart | Roche, Cat#: 2 239 264 |
| DNAMaster SYBR Green I | |
| Standard DNA | PCRII-E1A plasmid (4868 bp, conc.: |
| | 1.08 μg/μL) |
| Primers | |
| E1A-forword | 5′-CCT GTG TCT AGA GAA TGC AA-3′ |
| | (SEQ ID NO: 5) |
| E1A-reverse | 5′-ACA GCT CAA GTC CAA AGG TT-3′ |
| | (SEQ ID NO: 6) |
| Media | |
| H1299 | RPMI-1640: 500 mL |
| | FBS: 50 mL |
| | PG/SM: 5 mL |
| A549 | D-MEM F-12: 500 mL |
| | FBS: 50 mL |
| | PG/SM: 5 mL |
| Medium for antiviral assay | RPMI-1640: 500 mL |
| | FBS: 10 mL |

### 2. Experimental Design

### 1) XTT Assay For Measuring Cell Viability

### Cell Culture

Cells frozen in liquid nitrogen were thawed in water bath at 37° C., seeded at T25 flask, and cultured at 70-90% sub-confluent monolayer. Cells were detached with trypsin, and prepared suspension at a concentration of 1.0×10⁴ cells/ml.

Cells were seeded in 96 well-plate (100 μL/well, 1000 cells/well), then cultured with 5% CO₂ at 37° C.

### Virus Infection and CDV Treatment

Viruses

Viruses were diluted at MOI of 1 (OBP-301, OBP-202) in H1299 and 5 in A549 (PFU/cell). Medium in each well was removed, 150 μL of diluted virus solution was added to each well of the plate. 2 hours later, medium was aspirated and replaced with fresh medium containing 2% FCS and serially diluted CDV. Cells were incubated with 5% CO₂ at 37° C. for 7 days after infection.

Viruses were diluted at MOI of 0.1 (OBP-401) or 10 (ONYX-015) in H1299. Medium in each well was removed, 50 μL of diluted virus solution was added to each well of the plate. 2 hours later, medium was aspirated and replaced with fresh medium containing 2% FCS and serially diluted CDV. Cells were incubated with 5% CO₂ at 37° C. for 7 days after infection.

### XTT Assay

100 μL of PBS, 50 μL of XTT labeling reagent, and 1 μL of ion coupling reagent per well of plate were mixed.

Medium in each well of plate was removed, and 150 μL of mixture prepared above was added to each well. Plate was incubated with 5% CO₂ at 37° C. for 4 hours, and then calculated absorbance at 450 nm and 690 nm. Protection was determined by the formula below:

Protection(%)={OD(AdV(+):CDV(+))−OD(AdV(+):CDV(−))}/{OD(AdV(−):CDV(+))−OD(AdV(+):CDV(−))}×100.

### 2) Real-Time Quantitative PCR For Measuring Viral DNA Copy Number

### Cell Culture

Cells were seeded in 6-well plate (2 mL/well, 2×10⁵ cells/well), and incubated with 5% CO₂ at 37° C.

### Viral Infection and CDV Treatment

Viruses were diluted at a MOI of 10. Medium in each well was removed, 500 μL of diluted virus solution was added to each well of the plate. Plates were incubated with 5% CO₂ at 37° C. for 2 hours with gently shaking every 15 minutes. Medium was aspirated, washed with PBS twice, and replaced with fresh medium containing 2% FCS and serially diluted CDV. Cells were incubated with 5% CO₂ at 37° C. for 24 hours after infection.

### DNA Extraction and O-PCR Assay

Cells were detached with Trypsin-EDTA, and collected by centrifuging. Total DNA was extracted using QIAamp™ DNA Mini Kit according to manufacture's instructions with some modifications: addition of 20 μL of RNaseA in each sample when adding ProK. DNA was eluted by 50˜100 μL of D. W.

Concentration of DNA was determined by measuring O.D.₂₆₀. Each DNA samples was diluted by D. W. at a concentration of 5 ng/μL.

Master-mix for E1A primer was prepared using LightCycler™ Faststart DNAMaster SYBR Green™ according to Table 1. Standard DNA was prepared by serially 10 times dilution of pCRII-E1A plasmid (1×10⁹, 1×10⁸, 1×10⁷, 1×10⁶, 1×10⁵, 1×10⁴, and 1×10³ copies/μL). 2 μL of Sample or Standard DNA was and 18 μL of Master mix were added into capillaries (total volume was 20 μL per capillary).

TABLE 1
Reaction mix for E1A quantification

| Reagent | Stock Conc. | Volume (μL) | Final |
|---|---|---|---|
| H₂O | — | 12.4 | — |
| MgCl₂ | 25 mM | 1.6 | 3.0 mM |
| Forward Primer | 10 μM | 1.0 | 0.5 μM |
| Reverse Primer | 10 μM | 1.0 | 0.5 μM |
| LC Faststart DNA Master SYBR | 10 X | 2.0 | 1 X |
| Green I | | | |
| total | | 18.0 | |

Reaction Vol.: 20 μL (Mix 18 μL + Sample 2 μL)

Q-PCR was performed according to the program of Table 2 (LightCycler quicksystem 350S). Results obtained here were analyzed using LightCycler softwere.

TABLE 2
Program of LightCycler for E1A quantification

| | | | | | |
|---|---|---|---|---|---|
| Program | Denaturation | Type | None | Cycles | 1 |

| | | | | 2nd | | | |
|---|---|---|---|---|---|---|---|
| | | Hold | | Target | Step | Step | |
| Segment | Temperature | Time | Slope | Temp | size | Delay | Acquisition |
| Number | Target (° C.) | (sec) | (° C./sec) | (° C.) | (° C.) | (Cycles) | Mode |
| 1 | 95 | 600 | 20 | 0 | 0 | 0 | None |

| | | | | | |
|---|---|---|---|---|---|
| | | | Quanti- | | |
| Program | Amplification | Type | fication | Cycles | 40 |

| | | | | 2nd | | | |
|---|---|---|---|---|---|---|---|
| | | Hold | | Target | Step | Step | |
| Segment | Temperature | Time | Slope | Temp | size | Delay | Acquisition |
| Number | Target (° C.) | (sec) | (° C./sec) | (° C.) | (° C.) | (Cycles) | Mode |
| 1 | 95 | 10 | 20 | 0 | 0 | 0 | None |
| 2 | 60 | 15 | 20 | 0 | 0 | 0 | None |
| 3 | 72 | 8 | 20 | 0 | 0 | 0 | Single |

| | | | | | |
|---|---|---|---|---|---|
| | | | Melting | | |
| Program | melting | Type | Curves | Cycles | 1 |

| | | | | 2nd | | | |
|---|---|---|---|---|---|---|---|
| | | Hold | | Target | Step | Step | |
| Segment | Temperature | Time | Slope | Temp | size | Delay | Acquisition |
| Number | Target (° C.) | (sec) | (° C./sec) | (° C.) | (° C.) | (Cycles) | Mode |
| 1 | 95 | 0 | 20 | 0 | 0 | 0 | None |
| 2 | 70 | 15 | 20 | 0 | 0 | 0 | None |
| 3 | 95 | 0 | 0.1 | 0 | 0 | 0 | Continuous |

| | | | | | |
|---|---|---|---|---|---|
| Program | cooling | Type | None | Cycles | 1 |

| | | | | 2nd | | | |
|---|---|---|---|---|---|---|---|
| | | Hold | | Target | Step | Step | |
| Segment | Temperature | Time | Slope | Temp | size | Delay | Acquisition |
| Number | Target (° C.) | (sec) | (° C./sec) | (° C.) | (° C.) | (Cycles) | Mode |
| 1 | 40 | 30 | 20 | 0 | 0 | 0 | None |

| | | |
|---|---|---|
| Setting the Fluorescence Parameters | Display Mode | F1/1 |

### Example 2

### Telomelysin (OBP-301)

We have tested whether CDV inhibits the cytopathic effect of Teleomelysin in vitro. We measured the cell viability of H1299 and A549 cells treated with Telomelysin in the presence or absence of CDV by XTT assay (see Table 3A, 3B). As shown in FIG. 1A, although CDV itself was toxic in both cell lines at high concentrations, CDV apparently inhibited Telomelysin-mediated cytopathic effect. EC₅₀, CDV concentration that archive the protection to 50%, were calculated as 20.4 μM in H1299 and 35.9 μM in A549 cells, whereas the CC₅₀, CDV concentration that reduce the relative cell viability to 50%, were 146.4 μM in H1299 cells and 106.9 μM in A549 cells.

We have also tested whether CDV inhibits the viral replication by real-time quantitative PCR assay in H1299 cells (FIG. 1B, Table 3C). Viral E1A copy number was reduced in the cells infected both in Telomelysin or wild-type AdV by CDV treatment in a dose-dependent manner. EC₅₀ (E1A), CDV concentration that reduces the relative E1A copy number of Telomelysin to 50%, was calculated as 19.55 μM.

These results suggest that CDV inhibits the oncolytic activity of Telomelysin by inhibiting its DNA replication. These result also suggest that CDV treats the infection of Telomelysin in a mammal.

TABLE 3A
Data of XTT assay in H1299 cells (Telomelysin)

| | μM | O.D. | | | | Ave. | Relative cell viability | | | | Ave. | S.D. |
|---|---|---|---|---|---|---|---|---|---|---|---|---|
| CDV | 0 | 1.269 | 1.198 | 1.246 | 1.129 | 1.211 | 1.048 | 0.990 | 1.029 | 0.933 | 1.000 | 0.051 |
| | 0.32 | 1.299 | 1.417 | 1.316 | 1.166 | | 1.073 | 1.171 | 1.087 | 0.963 | 1.074 | 0.085 |
| | 1.6 | 1.262 | 1.344 | 1.144 | 1.174 | | 1.043 | 1.110 | 0.945 | 0.970 | 1.017 | 0.075 |
| | 8 | 1.255 | 1.262 | 1.267 | 1.104 | | 1.037 | 1.043 | 1.047 | 0.912 | 1.010 | 0.065 |
| | 40 | 1.113 | 1.060 | 1.082 | 1.215 | | 0.919 | 0.876 | 0.894 | 1.004 | 0.923 | 0.057 |
| | 200 | 0.509 | 0.438 | 0.497 | 0.485 | | 0.420 | 0.362 | 0.411 | 0.401 | 0.398 | 0.026 |
| | μM | O.D. | | | | Ave. | Protection (%) | | | | Ave. | S.D. |
| CDV + Telomelysin | 0 | 0.112 | 0.099 | 0.151 | 0.108 | 0.118 | −0.503 | −1.693 | 3.065 | −0.869 | 0.000 | 2.103 |
| | 0.32 | 0.245 | 0.161 | 0.183 | 0.230 | | 11.665 | 3.980 | 5.993 | 10.293 | 7.983 | 3.600 |
| | 1.6 | 0.053 | 0.130 | 0.145 | 0.154 | | −5.901 | 1.144 | 2.516 | 3.339 | 0.274 | 4.216 |
| | 8 | 0.137 | 0.311 | 0.106 | 0.050 | | 1.784 | 17.704 | −1.052 | −6.176 | 3.065 | 10.300 |
| | 40 | 0.905 | 1.046 | 1.102 | 1.082 | | 72.049 | 84.950 | 90.073 | 88.243 | 83.829 | 8.134 |
| | 200 | 0.455 | 0.434 | 0.426 | 0.439 | | 30.878 | 28.957 | 28.225 | 29.414 | 29.369 | 1.119 |

TABLE 3B
Data of XTT assay of A549 cells (Telomelysin)

| | μM | O.D. | | | | Ave. | Relative cell viability | | | | Ave. | S.D. |
|---|---|---|---|---|---|---|---|---|---|---|---|---|
| CDV | 0 | 1.884 | 1.829 | 1.937 | 1.998 | 1.912 | 0.985 | 0.957 | 1.013 | 1.045 | 1.000 | 0.038 |
| | 10 | 1.793 | 1.840 | 1.878 | 1.899 | | 0.938 | 0.962 | 0.982 | 0.993 | 0.969 | 0.024 |
| | 20 | 1.676 | 1.766 | 1.832 | 1.877 | | 0.877 | 0.924 | 0.958 | 0.982 | 0.935 | 0.046 |
| | 30 | 1.688 | 1.685 | 1.795 | 1.805 | | 0.883 | 0.881 | 0.939 | 0.944 | 0.912 | 0.034 |
| | 40 | 1.644 | 1.740 | 1.742 | 1.819 | | 0.860 | 0.910 | 0.911 | 0.951 | 0.908 | 0.037 |
| | 50 | 1.610 | 1.649 | 1.691 | 1.565 | | 0.842 | 0.862 | 0.884 | 0.819 | 0.852 | 0.028 |
| | 60 | 1.515 | 1.546 | 1.601 | 1.443 | | 0.792 | 0.809 | 0.837 | 0.755 | 0.798 | 0.034 |
| | 70 | 1.345 | 1.429 | 1.374 | 1.356 | | 0.703 | 0.747 | 0.719 | 0.709 | 0.720 | 0.020 |
| | 80 | 1.352 | 1.410 | 1.306 | 1.254 | | 0.707 | 0.737 | 0.683 | 0.656 | 0.696 | 0.035 |
| | 90 | 1.055 | 1.158 | 0.991 | 0.959 | | 0.552 | 0.606 | 0.518 | 0.502 | 0.544 | 0.046 |
| | 100 | 0.863 | 0.921 | 0.838 | 0.977 | | 0.451 | 0.482 | 0.438 | 0.511 | 0.471 | 0.032 |
| | μM | O.D. | | | | Ave. | Protection (%) | | | | Ave. | S.D. |
| CDV + Telomelysin | 0 | 0.036 | 0.030 | 0.046 | 0.051 | 0.041 | −0.254 | −0.574 | 0.281 | 0.548 | 0.000 | 0.508 |
| | 10 | 0.048 | 0.062 | 0.078 | 0.078 | | 0.387 | 1.136 | 1.991 | 1.991 | 1.376 | 0.773 |
| | 20 | 0.122 | 0.179 | 0.244 | 0.182 | | 4.342 | 7.388 | 10.862 | 7.548 | 7.535 | 2.664 |
| | 30 | 0.666 | 0.868 | 0.817 | 0.813 | | 33.413 | 44.208 | 41.483 | 41.269 | 40.094 | 4.650 |
| | 40 | 1.016 | 1.049 | 1.143 | 1.200 | | 52.118 | 53.881 | 58.904 | 61.951 | 56.713 | 4.523 |
| | 50 | 1.054 | 1.212 | 1.136 | 1.342 | | 54.148 | 62.592 | 58.530 | 69.539 | 61.202 | 6.540 |
| | 60 | 1.234 | 1.284 | 1.363 | 1.319 | | 63.768 | 66.440 | 70.661 | 68.310 | 67.295 | 2.918 |
| | 70 | 1.134 | 1.198 | 1.232 | 1.280 | | 58.424 | 61.844 | 63.661 | 66.226 | 62.538 | 3.280 |
| | 80 | 1.007 | 1.150 | 1.187 | 1.066 | | 51.637 | 59.279 | 61.256 | 54.790 | 56.740 | 4.347 |
| | 90 | 0.986 | 0.994 | 0.908 | 1.003 | | 50.514 | 50.942 | 46.346 | 51.423 | 49.806 | 2.336 |
| | 100 | 0.819 | 0.730 | 0.789 | 0.676 | | 41.590 | 36.834 | 39.987 | 33.948 | 38.090 | 3.395 |

TABLE 3C
Data of PCR analysis in H1299 cells (Telomelysin)

| | | |
|---|---|---|
| μM | E1A copy number/ng DNA | % |

Telomelysin + CDV

| | | |
|---|---|---|
| 0 | 1.497E+08 | 100.00 |
| 0.16 | 1.461E+08 | 97.60 |
| 0.8 | 1.481E+08 | 98.93 |
| 4 | 1.367E+08 | 91.32 |
| 20 | 7.046E+07 | 47.07 |
| 100 | 1.496E+07 | 9.99 |

Wt-Ad + CDV

| | | |
|---|---|---|
| 0 | 5.236E+07 | 100.00 |
| 0.16 | 6.152E+07 | 117.49 |
| 0.8 | 5.008E+07 | 95.65 |
| 4 | 3.911E+07 | 74.69 |
| 20 | 1.719E+07 | 32.83 |
| 100 | 2.509E+06 | 4.79 |

### Example 3

### TelomeScan (OBP-401), ONYX-015

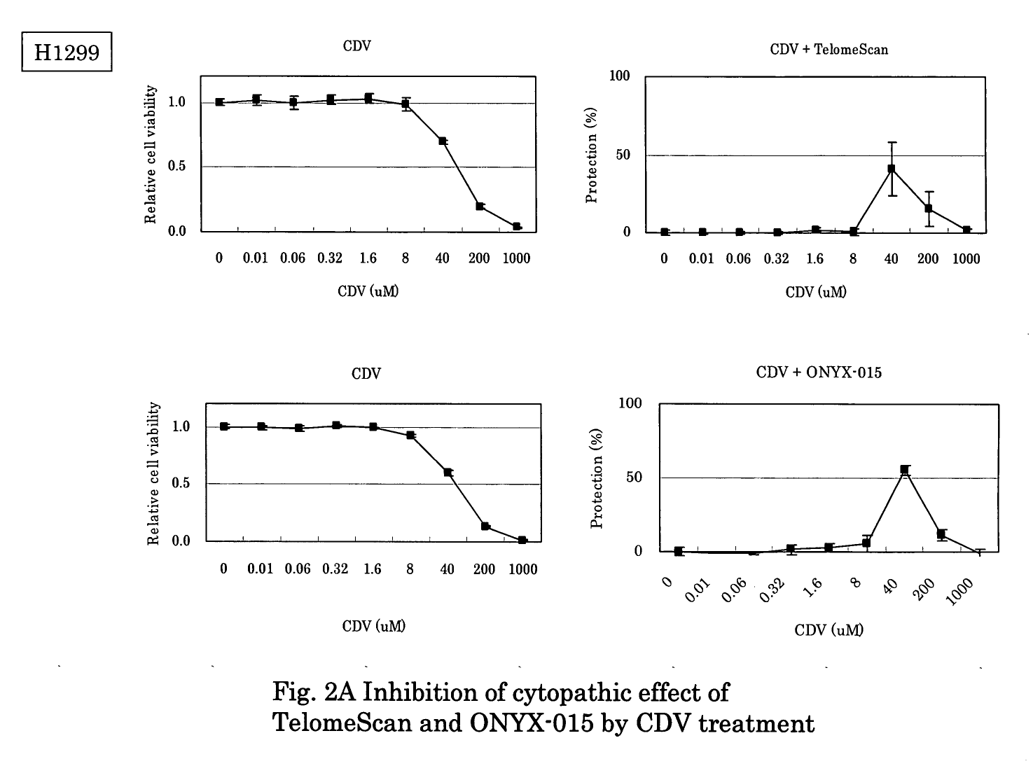

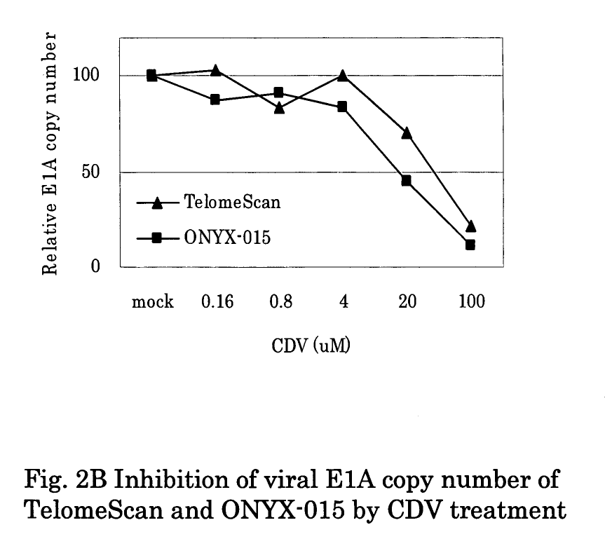

We have further tested whether CDV inhibits the cytopathic effect of TeleomeScan and ONYX-015 in vitro. We measured the cell viability of H1299 cells treated with TelomeScan or ONYX-015 in the presence or absence of CDV by XTT assay (see Table 4A, 4B). As shown in FIG. 2A, although CDV itself was toxic in both cell lines at high concentrations, CDV apparently inhibited the cytopathic effect induced by TelomeScan or ONYX-015.

We have also tested whether CDV inhibits the viral replication by real-time quantitative PCR assay in H1299 cells (FIG. 2B, Table 4C). Viral E1A copy number was reduced in the cells infected both in TelomeScan or ONYX-015 by CDV treatment in a dose-dependent manner.

These results suggest that CDV inhibits the oncolytic activity both of TelomeScan and ONYX-015 by inhibiting its DNA replication. These results also suggest that CDV treats the infection of TelomeScan or ONYX-015 in a mammal.

TABLE 4A
Data of XTT assay (OBP-401)

| | μM | O.D. | | | | Ave. | Relative cell viability | | | | Ave. | SD |
|---|---|---|---|---|---|---|---|---|---|---|---|---|
| CDV | 0 | 2.310 | 2.281 | 2.221 | 2.199 | 2.253 | 1.025 | 1.013 | 0.986 | 0.976 | 1.000 | 0.023 |
| | 0.01 | 2.407 | 2.258 | 2.272 | 2.203 | | 1.068 | 1.002 | 1.009 | 0.978 | 1.014 | 0.038 |
| | 0.06 | 2.410 | 2.247 | 2.190 | 2.128 | | 1.070 | 0.997 | 0.972 | 0.945 | 0.996 | 0.054 |
| | 0.32 | 2.407 | 2.256 | 2.324 | 2.202 | | 1.068 | 1.001 | 1.032 | 0.977 | 1.020 | 0.039 |
| | 1.6 | 2.391 | 2.352 | 2.327 | 2.211 | | 1.061 | 1.044 | 1.033 | 0.981 | 1.030 | 0.034 |
| | 8 | 2.302 | 2.288 | 2.250 | 2.068 | | 1.022 | 1.016 | 0.999 | 0.918 | 0.989 | 0.048 |
| | 40 | 1.524 | 1.546 | 1.591 | 1.598 | | 0.677 | 0.686 | 0.706 | 0.709 | 0.695 | 0.016 |
| | 200 | 0.457 | 0.417 | 0.418 | 0.498 | | 0.203 | 0.185 | 0.186 | 0.221 | 0.199 | 0.017 |
| | 1000 | 0.096 | 0.082 | 0.084 | 0.075 | | 0.043 | 0.036 | 0.037 | 0.033 | 0.037 | 0.004 |
| | μM | O.D. | | | | Ave. | Protection (%) | | | | Ave. | SD |
| CDV + OBP- | 0 | 0.081 | 0.035 | 0.101 | 0.044 | 0.065 | 0.720 | −1.383 | 1.634 | −0.971 | 0.000 | 1.420 |
| 401 | 0.01 | 0.072 | 0.060 | 0.067 | 0.042 | | 0.309 | −0.240 | 0.080 | −1.063 | −0.229 | 0.600 |
| | 0.06 | 0.054 | 0.100 | 0.056 | 0.056 | | −0.514 | 1.589 | −0.423 | −0.423 | 0.057 | 1.022 |
| | 0.32 | 0.080 | 0.074 | 0.048 | 0.074 | | 0.674 | 0.400 | −0.789 | 0.400 | 0.171 | 0.653 |
| | 1.6 | 0.064 | 0.122 | 0.113 | 0.131 | | −0.057 | 2.594 | 2.183 | 3.006 | 1.931 | 1.368 |
| | 8 | 0.071 | 0.049 | 0.045 | 0.137 | | 0.263 | −0.743 | −0.926 | 3.280 | 0.469 | 1.946 |
| | 40 | 1.482 | 0.707 | 0.689 | 0.964 | | 64.766 | 29.337 | 28.514 | 41.086 | 40.926 | 16.899 |
| | 200 | 0.444 | 0.516 | 0.582 | 0.045 | | 17.314 | 20.606 | 23.623 | −0.926 | 15.154 | 11.025 |
| | 1000 | 0.077 | 0.143 | 0.094 | 0.075 | | 0.537 | 3.554 | 1.314 | 0.446 | 1.463 | 1.448 |

TABLE 4B
Data of XTT assay (ONYX-015)

| | μM | O.D. | | | | Ave. | Relative cell viability | | | | Ave. | SD |
|---|---|---|---|---|---|---|---|---|---|---|---|---|
| CDV | 0 | 2.037 | 1.980 | 2.041 | 1.988 | 2.012 | 1.013 | 0.984 | 1.015 | 0.988 | 1.000 | 0.016 |
| | 0.01 | 2.031 | 2.009 | 1.992 | 1.951 | | 1.010 | 0.999 | 0.990 | 0.970 | 0.992 | 0.017 |
| | 0.06 | 2.050 | 1.991 | 1.974 | 1.911 | | 1.019 | 0.990 | 0.981 | 0.950 | 0.985 | 0.028 |
| | 0.32 | 2.028 | 2.052 | 2.007 | 2.018 | | 1.008 | 1.020 | 0.998 | 1.003 | 1.007 | 0.010 |
| | 1.6 | 2.003 | 1.977 | 2.006 | 1.980 | | 0.996 | 0.983 | 0.997 | 0.984 | 0.990 | 0.008 |
| | 8 | 1.881 | 1.867 | 1.857 | 1.836 | | 0.935 | 0.928 | 0.923 | 0.913 | 0.925 | 0.009 |
| | 40 | 1.155 | 1.263 | 1.225 | 1.183 | | 0.574 | 0.628 | 0.609 | 0.588 | 0.600 | 0.024 |
| | 200 | 0.258 | 0.292 | 0.269 | 0.272 | | 0.128 | 0.145 | 0.134 | 0.135 | 0.136 | 0.007 |
| | 1000 | 0.027 | 0.025 | 0.030 | 0.042 | | 0.013 | 0.012 | 0.015 | 0.021 | 0.015 | 0.004 |
| | μM | O.D. | | | | Ave. | Protection (%) | | | | Ave. | SD |
| CDV + ONYX- | 0 | 0.131 | 0.087 | 0.027 | 0.015 | 0.065 | 3.391 | 1.130 | −1.952 | −2.569 | 0.000 | 2.780 |
| 015 | 0.01 | 0.041 | 0.049 | 0.045 | 0.040 | | −1.233 | −0.822 | −1.027 | −1.284 | −1.092 | 0.211 |
| | 0.06 | 0.061 | 0.066 | 0.030 | 0.053 | | −0.205 | 0.051 | −1.798 | −0.616 | −0.642 | 0.818 |
| | 0.32 | 0.156 | 0.061 | 0.033 | 0.128 | | 4.675 | −0.205 | −1.644 | 3.237 | 1.516 | 2.938 |
| | 1.6 | 0.101 | 0.044 | 0.172 | 0.149 | | 1.849 | −1.079 | 5.497 | 4.315 | 2.646 | 2.911 |
| | 8 | 0.256 | 0.264 | 0.021 | 0.163 | | 9.812 | 10.223 | −2.260 | 5.035 | 5.703 | 5.808 |
| | 40 | 1.212 | 1.146 | 1.161 | 1.056 | | 58.926 | 55.536 | 56.306 | 50.912 | 55.420 | 3.337 |
| | 200 | 0.394 | 0.247 | 0.234 | 0.262 | | 16.902 | 9.350 | 8.682 | 10.121 | 11.264 | 3.805 |
| | 1000 | 0.021 | 0.019 | 0.139 | 0.020 | | −2.260 | −2.363 | 3.802 | −2.312 | −0.783 | 3.057 |

TABLE 4C
Data of real-time quantitative PCR analysis

| | | |
|---|---|---|
| uM | E1A copy number/ng DNA | % |

TelomeScan + CDV

| | | |
|---|---|---|
| 0 | 2.271E+08 | 100.00 |
| 0.16 | 2.350E+08 | 103.48 |
| 0.8 | 1.890E+08 | 83.22 |
| 4 | 2.285E+08 | 100.62 |
| 20 | 1.594E+08 | 70.19 |
| 100 | 4.957E+07 | 21.83 |

ONYX-015 + CDV

| | | |
|---|---|---|
| 0 | 2.634E+07 | 100.00 |
| 0.16 | 2.291E+07 | 86.98 |
| 0.8 | 2.398E+07 | 91.04 |
| 4 | 2.205E+07 | 83.71 |
| 20 | 1.188E+07 | 45.10 |
| 100 | 2.950E+06 | 11.20 |

### REFERENCE

1) U.S. Pat. No. 5,142,051 N-Phosphonylmethoxyalkyl derivatives of Pyrimidine and Purine Bases and a Therapeutical Composition Therefrom with Antiviral Activity
2) Comparison of HSV-1 thymidine kinase-dependent and -independent inhibition of replication-competent adenoviral vectors by a panel of drugs Oliver Wildner, et al. Cancer Gene Therapy (2003) volume 10, 791-802
3) Oncolytic viral therapies—the clinical experience Manish Aghi, et al. Oncogene (2005) volume 24, 7802-7816
4) Intracellular Metabolism of the Antiherpesvirus Agent (S)-1-[3-hydroxy-2-(phosphonylmethoxy)propyl]cytosine. Ho H T, Woods K L, Bronson J J, De Boeck H, Martin J C and Hitchcock M J M. Mol Pharmacol 41:197-202, 1992.
5) Interaction Between the Diphosphate of (S)-1-(3-hydroxy-2-phosphonylmethoxypropyl)cytosine, zalcitabineTP, zidovudineTP, and FIAUTP with Human DNA Polymerases b and g. Cherrington J M, Allen S J W, McKee B H, and Chen M S. Kinetic Analysis of the Biochem Pharmacol 48:1986-1988, 1994.
6) Interaction of Cidofovir Diphosphate with Human Cytomegalovirus DNA Polymerase.
   Xiong X, Smith J L, Kim C, Huang E, and Chen M S. Kinetic Analysis of the Biochem Pharmacol 51:1563-1567, 1996.

What is claimed is:

1. A method for inhibiting an oncolytic virus activity, comprising contacting a cidofovir with the virus.

2. A method according to claim 1, wherein said oncolytic virus is oncolytic adenovirus.

3. A method according to claim 2, wherein said oncolytic adenovirus is one selected from the group consisting of Telomelysin, Telomelysin-GFP and ONYX-015.

4. A method for treating an oncolytic viral infection in an individual, comprising administering to said individual an effective amount of cidofovir.

5. A method according to claim 4, wherein said individual is a cancer patient.

6. A method according to claim 4, wherein said oncolytic virus is oncolytic adenovirus.

7. A method according to claim 6, wherein said oncolytic adenovirus is one selected from the group consisting of Telomelysin, Telomelysin-GFP and ONYX-015.

8. A method for preventing or suppressing an oncolytic viral replication in an individual, comprising administering to said mammal an effective amount of cidofovir.

9. A method according to claim 8, wherein said individual is a cancer patient.

10. A method according to claim 8, wherein said oncolytic virus is oncolytic adenovirus.

11. A method according to claim 10, wherein said oncolytic adenovirus is one selected from the group consisting of Telomelysin, Telomelysin-GFP and ONYX-015.


SEQUENCE LISTING (6 sequences)

SEQ ID NO: 1 - DNA, 455 bp, homo sapiens
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 2 - DNA, 899 bp, adenovirus
acaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtg

SEQ ID NO: 3 - DNA, 1823 bp, adenovirus
ctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggc

SEQ ID NO: 4 - DNA, 621 bp, adenovirus
tgcatctagggcggccaattccgcccctctcccccccccccctctccctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgcca

SEQ ID NO: 5 - DNA, 20 bp, Artificial
  synthetic DNA
cctgtgtctagagaatgcaa

SEQ ID NO: 6 - DNA, 20 bp, Artificial
  synthetic DNA
acagctcaagtccaaaggtt